(19) United States
(12) Patent Application Publication
(10) Pub. No.: US 2021/0012890 A1
(43) Pub. Date: Jan. 14, 2021

(54) HEALTH CARE DELIVERY SYSTEM AND METHOD

(71) Applicant: Tenfold Health, LLC, Bend, OR (US)

(72) Inventors: James M. HENRY, IV, Bend, OR (US); Michael J. BONETTO, Bend, OR (US)

(21) Appl. No.: 17/035,676

(22) Filed: Sep. 28, 2020

Related U.S. Application Data
(63) Continuation of application No. 15/703,058, filed on Sep. 13, 2017, now abandoned.
(60) Provisional application No. 62/394,152, filed on Sep. 13, 2016.

(51) Int. Cl. G16H 40/20

(52) U.S. Cl. CPC G16H 40/20

(57) ABSTRACT

A health care delivery system for a health care organization that has health care service providers, and includes plural programs for delivering health care services, includes a mechanism for identifying root causes of disease and health conditions. Also includes is a mechanism for researching and identifying the best practice for public health interventions, and a mechanism for prioritizing and integrating efforts of health care service providers across the entire health care organization. The system also includes a mechanism for directing oversight of programs, and there is also a method of delivering health care that uses steps that correspond to the above mechanisms.

### CROSS-REFERENCE TO RELATED APPLICATIONS

This application is a continuation of U.S. patent application Ser. No. 15/703,058, filed Sep. 13, 2017 and entitled HEALTH CARE DELIVERY SYSTEM AND METHOD, which application claims the benefit of U.S. Provisional Patent Application No. 62/394,152, filed Sep. 13, 2016 and entitled “HEALTH CARE DELIVERY SYSTEM AND METHOD”, which application is incorporated herein by reference for all purposes.

### FIELD OF THE INVENTION

The disclosure relates to systems and methods for delivery of health care services.

### SUMMARY

In one embodiment, the invention is a health care delivery system for a health care organization that has health care service providers, and includes plural programs for delivering health care services. That embodiment includes a mechanism for identifying root causes of disease and health conditions. Also included is a mechanism for researching and identifying the best practice for public health interventions, and a mechanism for prioritizing and integrating efforts of health care service providers across the entire health care organization. The system also includes a mechanism for directing oversight of programs.

In another embodiment, the invention is a method of delivering health care for a health care organization that has health care service providers, and includes plural programs for delivering health care services. That embodiment includes identifying root causes of disease and health conditions. Also included is a step of researching and identifying the best practice for public health interventions, and a step of prioritizing and integrating efforts of health care service providers across the entire health care organization. The method also includes a step of directing oversight of programs.

### DETAILED DESCRIPTION

Attachment A provides a detailed description of the invention.

What is claimed is:

1. A health care delivery system for a health care organization that has health care service providers, and includes plural programs for delivering health care services, comprising:
a mechanism for identifying root causes of disease and health conditions;
a mechanism for researching and identifying the best practice for public health interventions;
a mechanism for prioritizing and integrating efforts of health care service providers across the entire health care organization; and
a mechanism for directing oversight of programs.

2. A method of delivering health care for a health care organization that has health care service providers, and includes plural programs for delivering health care services, comprising:
identifying root causes of disease and health conditions;
researching and identifying the best practice for public health interventions;
prioritizing and integrating efforts of health care service providers across the entire health care organization; and
directing oversight of programs.
